(19) United States
(12) Patent Application Publication
(10) Pub. No.: US 2024/0279069 A1
(43) Pub. Date: Aug. 22, 2024

(54) TWO-DIMENSIONAL PARTICLE, CONDUCTIVE FILM, CONDUCTIVE PASTE, AND METHOD FOR PRODUCING TWO-DIMENSIONAL PARTICLE

(71) Applicants: Murata Manufacturing Co., Ltd., Nagaokakyo-shi (JP); Drexel University, Philadelphia, PA (US)

(72) Inventors: Mikhail SHEKHIREV, Philadelphia, PA (US); Christopher Eugene SHUCK, Philadelphia, PA (US); Yury GOGOTSI, Ivyland, PA (US); Yuusuke OGAWA, Nagaokakyo-shi (JP); Masashi KOYANAGI, Nagaokakyo-shi (JP)

(21) Appl. No.: 18/610,823

(22) Filed: Mar. 20, 2024

Related U.S. Application Data
(63) Continuation of application No. PCT/JP2022/034785, filed on Sep. 16, 2022.
(60) Provisional application No. 63/247,973, filed on Sep. 24, 2021.

(51) Int. Cl. C01B 32/907; A61B 5/263; B01J 20/02; B01J 20/28; B01J 20/30; C08K 9/02; C09D 5/24; C09D 7/40; C09D 7/62; H01B 1/22; H05K 9/00

(52) U.S. Cl. CPC C01B 32/907; A61B 5/263; B01J 20/0211; B01J 20/0248; B01J 20/0274; B01J 20/28007; B01J 20/28011; B01J 20/28016; B01J 20/3071; B01J 20/3085; C09D 5/24; C09D 7/62; C09D 7/69; C09D 7/70; H01B 1/22; H05K 9/0083; C01P 2002/52; C01P 2004/20; C01P 2004/61; C01P 2006/40; C08K 9/02; C08K 2201/001; C08K 2201/003; C08K 2201/004; C08K 2201/011

(57) ABSTRACT

A two-dimensional particle including: one or plural layers, the one or plural layers having a layer body represented by: MₘXₙ, wherein M is at least one metal of Group 3, 4, 5, 6, or 7, X is a carbon atom, a nitrogen atom, or a combination thereof, n is 1 to 4, m is more than n but not more than 5, and a modifier or terminal T existing on a surface of the layer body, wherein T is at least one selected from a hydroxyl group, an amine group, a fluorine atom, a chlorine atom, a bromine atom, an iodine atom, an oxygen atom, a sulfur atom, a selenium atom, a tellurium atom and a hydrogen atom; a metal cation is at least one cation selected from Na and K, and a content of Li in the two-dimensional particle is less than 0.002% by mass.

### CROSS REFERENCE TO RELATED APPLICATIONS

The present application is a continuation of International application No. PCT/JP2022/034785, filed Sep. 16, 2022, which claims priority to United States Provisional Patent Application No. 63/247,973, filed Sep. 24, 2021, the entire contents of each of which are incorporated herein by reference.

### TECHNICAL FIELD

The present invention relates to a two-dimensional particle, a conductive film, a conductive paste, and a method for producing a two-dimensional particle.

### BACKGROUND ART

In recent years, MXenes have been attracting attention as a new material with high conductivity. MXenes are a family of so-called two-dimensional material, and as will be described later, is a layered material in a form of one or plural layers. In general, MXenes can be in a form of particles (which can include powders, flakes, nanosheets, and the like) of such a layered material or assembled into arrays (films, fibers, coatings, composites, etc.).

Currently, various studies are being conducted toward the application of MXene to various electrical devices. For the above application, s required to further enhance the conductivity of a material containing MXene. As a part of the studies, a delamination treatment method has been studied for MXene to be obtained as an intercalated multilayered product or single-flake colloid.

Non Patent Literature 1 discloses that the amount of Li between MXene layers can be controlled by adding hydrochloric acid or the like to adjust the pH in a suspension prepared by intercalation using Li to about 2.9.

Non Patent Literature 2 discloses that delamination treatment of multilayer MXene was performed by handshaking with an existence of TMAOH (tetramethylammonium hydroxide).

In addition, Non Patent Literature 3 describes that a Li cation exists in an interlayer space of MXene due to LiCl used in chemical etching, and that a structural change of a powder occurs as a result of exchanging the Li cation with another metal ion.

Non Patent Literature 1: Hongwu Chen et al., “Pristine Titanium Carbide MXene Films with Environmentally Stable Conductivity and Superior Mechanical Strength” Advanced Functional Materials, 2020, 30, 1906996

Non Patent Literature 2: Mohamed Alhabeb et al., “Guidelines for Synthesis and Processing of Two-Dimensional Titanium Carbide (Ti3C2Tx MXene)” Chemistry of Materials, 2017, 29, 7633-7644

Non Patent Literature 3: Michael Ghidiu et al., “Ion-Exchange and Cation Solvation Reactions in Ti3C2 MXene” Chemistry of Materials, 2016, 28, 10, 3507-3514

### SUMMARY OF THE INVENTION

In the MXene described in Non Patent Literature 1, Li is not completely removed, and thus the conductivity decreases under high humidity conditions. In the MXene described in Non Patent Literature 2, TMAOH used for the delamination treatment of the multilayer MXene remains, the conductivity is low, and the conductivity further decreases due to moisture absorption, so that the reliability is not sufficiently satisfactory. In the MXene described in Non Patent Literature 3, the MXene remains as a multilayer MXene although Li cations have been exchanged with other metal ions, and hence the conductivity is low. Further, the MXene is a multilayer MXene, and hence it is not easy to form a conductive film.

An object of the present invention is to realize a two-dimensional particle capable of providing a conductive film capable of maintaining a high conductivity even under high humidity conditions. Another object of the present invention is to provide a method for producing such a two-dimensional particle.

The present invention includes the following aspects.

<1> A two-dimensional particle comprising:
one or plural players, the one or plural layers comprising a layer body represented by:

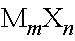

wherein M is at least one metal of Group 3, 4, 5, 6, or 7,
X is a carbon atom, a nitrogen atom, or a combination thereof,
n is not less than 1 and not more than 4, and
m is more than n but not more than 5,
and a modifier or terminal T existing on a surface of the layer body, wherein T is at least one selected from the group consisting of a hydroxyl group, an amine group, a fluorine atom, a chlorine atom, a bromine atom, an iodine atom, an oxygen atom, a sulfur atom, a selenium atom, a tellurium atom and a hydrogen atom; and
a metal cation comprising at least one cation selected from the group consisting of Na and K, wherein
a content of Li in the two-dimensional particle is less than 0.002% by mass.

<2> The two-dimensional particle according to <1>, wherein a content of chlorine atoms in the two-dimensional particle measured by combustion ion chromatography is 3% by mass or more in a sum of the layer and the metal cation.

<3> The two-dimensional particle according to <1> or <2>, wherein a total content of Na and K is not less than 0.1% by mass and not more than 10% by mass.

<4> The two-dimensional particle according to any one of <1> to <3>, wherein an average value of lengths of major axes of two-dimensional planes of the two-dimensional particle is not less than 1 μm and not more than 20 μm.

<5> The two-dimensional particle according to any one of <1> to <4>, wherein an average thickness of the two-dimensional particle is not less than 1 nm and not more than 10 nm.

<6> A conductive film comprising the two-dimensional particle according to any one of <1> to <5>.

<7> The conductive film according to <6>, wherein a conductivity of the conductive film is 2,000 S/cm or more.

<8> A conductive paste comprising the two-dimensional particle according to any one of <1> to <5> and a dispersion medium.

<9> A conductive composite comprising the two-dimensional particle according to any one of <1> to <5> and a resin.

<10> An electromagnetic shield comprising the two-dimensional particle according to any one of <1> to <5>.

<11> An adsorbent comprising the two-dimensional particle according to any one of <1> to <5>.

<12> A bioelectrode comprising the two-dimensional particle according to any one of <1> to <5>.

<13> A method for producing a two-dimensional particle, the method comprising:
(a) preparing a precursor represented by:

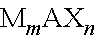

wherein M is at least one metal of Group 3, 4, 5, 6, or 7,
X is a carbon atom, a nitrogen atom, or a combination thereof,
A is at least one element of Group 12, 13, 14, 15, or 16,
n is not less than 1 and not more than 4, and
m is more than n but not more than 5;
(b) removing at least a part of A atoms from the precursor using an etching liquid to produce an etched product, wherein a concentration of chlorine atoms in the etching liquid is 10 mol/L or more
(c) washing the etched product with water to produce washed product;
(d) mixing the water washed product with a metal-containing compound to produce an intercalated product, wherein the metal-containing compound comprises at least one metal selected from the group consisting of Na and K;
(e) stirring the intercalated product to obtain a delaminated product; and
(f) washing the delaminated product with water to obtain a two-dimensional particle.

According to the present invention, a two-dimensional particle capable of providing a conductive film capable of maintaining a high conductivity even under high humidity conditions is realized. In addition, according to the present invention, a method for producing such a two-dimensional particle is provided.

### BRIEF DESCRIPTION OF THE DRAWINGS

FIGS. 1A and 1B are schematic cross-sectional views showing MXene particles of a layered material in one embodiment of the present invention, in which FIG. 1A shows a single-layer MXene particle and FIG. 1B shows a multilayer (exemplarily, two-layer) MXene particle.

FIG. 2 is a schematic cross-sectional view showing a conductive film according to one embodiment of the present invention.

### DETAILED DESCRIPTION OF THE PREFERRED EMBODIMENTS

### Embodiment 1: Two-Dimensional Particle

Hereinafter, the two-dimensional particle according to one embodiment of the present invention will be described in detail, but the present invention is not limited to such an embodiment.

The two-dimensional particle in the present embodiment is a two-dimensional particle of a layered material comprising one or plural layers, and comprising a metal cation.

The layer comprises a layer body represented by:

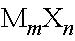

wherein M is at least one metal of Group 3, 4, 5, 6, or 7,
X is a carbon atom, a nitrogen atom, or a combination thereof, n is not less than 1 and not more than 4, and
m is more than n but not more than 5,
(the layer body can have a crystal lattice in which each X is located in an octahedral array of M),
and a modifier or terminal T existing on a surface of the layer body (more specifically, on at least one of both surfaces, facing each other, of the layer body), wherein T is at least one selected from the group consisting of a hydroxyl group, an amine group, a fluorine atom, a chlorine atom, a bromine atom, an iodine atom, an oxygen atom, a sulfur atom, a selenium atom, a tellurium atom and a hydrogen atom,
and the modifier or terminal T comprises at least a chlorine atom.

The layered material can be understood as a layered compound and can also be represented by “MₘXₙTₛ”, wherein s is any number and traditionally x or z may be used instead of s. Typically, n can be 1, 2, 3, or 4, but is not limited thereto.

T is preferably at least one selected from the group consisting of a hydroxyl group, a chlorine atom, an oxygen atom, and a hydrogen atom.

In the above formula of MXene, M is preferably at least one selected from the group consisting of Ti, Zr, Hf, V, Nb, Ta, Cr, Mo, Sc, Y, W, and Mn, more preferably at least one selected from the group consisting of Ti, Zr, Hf, V, Nb, Ta, Cr, Mo and Mn, and even more preferably at least one selected from the group consisting of Ti, V, Cr, and Mo.

MXenes whose above formula MₘXₙ is expressed as below are known:
SC₂C, TizC, TizN, Zr₂C, Zr₂N, Hf₂C, Hf₂N, V₂C, V₂N, Nb₂C, Ta₂C, Cr₂C, Cr₂N, Mo₂C, MO_{1.3}C, Cr_{1.3}C, (Ti, V)₂C, (Ti, Nb)₂C, W₂C, W_{1.3}C, MO₂N, Nb_{1.3}C, MO_{1.3}Y_{0.6}C, wherein, “1.3” and “0.6” mean about 1.3 (=4/3) and about 0.6 (=2/3), respectively, or
Ti₃C₂, Ti₃N₂, Ti₃ (CN), Zr₃C₂, (Ti, V)₃C₂, (Ti₂Nb)C₂, (Ti₂Ta)C₂, (Ti₂Mn)C₂, Hf 3C₂, (Hf₂V)C₂, (Hf₂Mn)C₂, (V₂Ti)C₂, (Cr₂Ti)C₂, (Cr₂V)C₂, (Cr₂Nb)C₂, (Cr₂Ta)C₂, (Mo₂Sc)C₂, (MO₂Ti)C₂, (MO₂Zr)C₂, (MO₂Hf)C₂, (MO₂V)C₂, (MO₂Nb)C₂, (MO₂Ta)C₂, (W₂Ti)C₂, (W₂Zr)C₂, (W₂Hf)C₂, or
Ti₄N₃, V₄C₃, Nb₄C₃, (Ti, Nb)₄C₃, Ta₄C₃, (Nb, Zr)₄C₃, (Ti₂Nb₂)C₃, (Ti₂Ta₂)C₃, (V₂Ti₂)C₃, (V₂Nb₂)C₃, (V₂Ta₂)C₃, (Nb₂Ta₂)C₃, (Cr₂Ti₂)C₃, (Cr₂V₂)C₃, (Cr₂Nb₂)C₃, (Cr₂Ta₂)C₃, (MO₂Ti₂)C₃, (MO₂Zr₂)C₃, (MO₂Hf₂)C₃, (Mo, V)C₃, (MO₂Nb₂)C₃, (MO₂Ta₂)C₃, (W₂Ti₂)C₃, (W₂Zr₂)C₃, (W₂Hf₂)C₃, (MO_{2.7}V_{1.3})C₃, wherein “2.7” and “1.3” mean about 2.7 (=8/3) and about 1.3 (=4/3), respectively, or
(Mo, V)₅C₄.

Typically, in the above formula, M can be titanium or vanadium and X can be a carbon atom, a nitrogen atom, or both. For example, the MAX phase may be Ti₃AlC₂ and MXene may be Ti₃C₂Tₛ (in other words, M is Ti, X is C, n is 2, and m is 3).

It is noted, in the present invention, MXene may contain A atoms derived from the precursor MAX phase at a relatively small amount, for example, at 10% by mass or less with respect to the original amount of A atoms. The remaining amount of A atoms can be preferably 8% by mass or less, and more preferably 6% by mass or less. However, even if the remaining amount of A atoms exceeds 10% by mass, there may be no problem depending on the application and conditions of use of the two-dimensional particle.

In the present description, the layer may be referred to as an MXene layer, and the two-dimensional particle may be referred to as an MXene two-dimensional particle or an MXene particle.

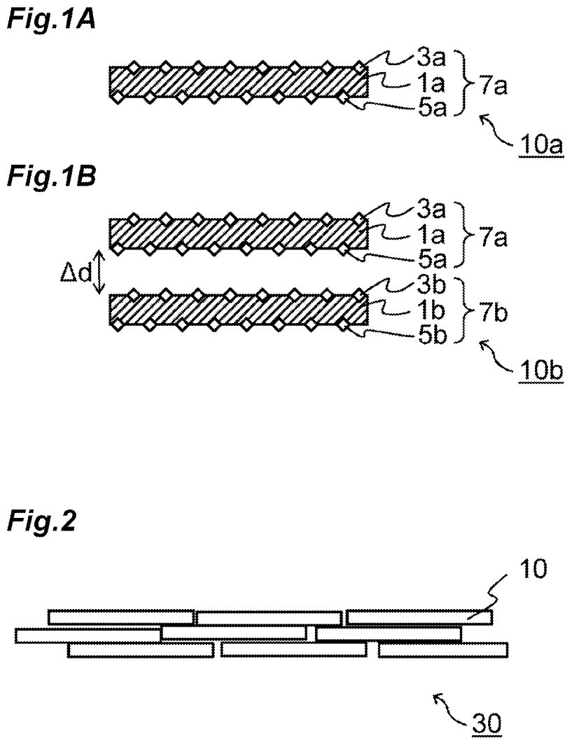

The two-dimensional particle of the present embodiment may be an aggregate comprising MXene particles with a single layer (hereinafter simply referred to as “MXene particles”) 10a (single-layer MXene particles) schematically illustrated in FIG. 1A. More specifically, the MXene particles 10a have layer bodies represented by MₘXₙ (MₘXₙ layers) 1a, and MXene layers 7a having modifiers or terminals T 3a, 5a existing on the surfaces of the layer bodies 1a (more specifically, on at least one of both surfaces, facing each other, of each layer). Therefore, the MXene layer 7a is also represented by “MₘXₙTₛ”, wherein s is any number. In FIG. 1A, metal cations are not shown.

The two-dimensional particle of the present embodiment may comprise one or plural layers. Examples of the MXene particle with plural layers (multilayer MXene particle) include, but are not limited to, an MXene particle 10b with two layers as schematically illustrated in FIG. 1B. 1b, 3b, 5b, and 7b in FIG. 1B are the same as 1a, 3a, 5a, and 7a in FIG. 1A described above. Two adjacent MXene layers (e.g., 7a and 7b) in the multilayer MXene particle may not necessarily be completely separated from each other, but may be partially in contact with each other. The MXene particle 10a may be one that exists as a single layer resulting from the multilayer MXene particle 10b being separated from one another, and may exist as a mixture of the single-layer MXene particles 10a and the multilayer MXene particles 10b in which some multilayer MXene particles 10b that are not separated remain. In FIG. 1B, metal cations are not shown.

Although not limiting the present embodiment, the thickness of each layer contained in the MXene particle (which corresponds to the MXene layers 7a, 7b) is, for example, not less than 0.8 nm and not more than 5 nm, and particularly not less than 0.8 nm and not more than 3 nm (which can vary mainly depending on the number of M atom layers included in each layer). For individual laminates of the multilayer MXene particles that may be included, the inter-layer distance (or gap dimension, denoted as Δd in FIG. 1B) is, for example, not less than 0.8 nm and not more than 10 nm, particularly not less than 0.8 nm and not more than 5 nm, and more particularly about 1 nm, and the total number of layers may be not less than 2 and not more than 20,000.

In the two-dimensional particle of the present embodiment, the multilayer MXene particle that may be contained is preferably an MXene particle having a small number of layers obtained through delamination treatment. The “small number of layers” means, for example, that the number of stacked MXene layers is 6 or less. The thickness in the stacking direction of the multilayer MXene particle having a small number of layers is preferably within 15 nm, and more preferably 10 nm or less. Hereinafter, the “multilayer MXene particle having a small number of layers” may be referred to as “few-layer MXene particle”. The single-layer MXene particle and the few-layer MXene particle may be collectively referred to as “single-layer/few-layer MXene particles”.

The two-dimensional particle of the present embodiment preferably comprises single-layer MXene particles and few-layer MXene particles, that is, single-layer/few-layer MXene particles. In the two-dimensional particle of the present embodiment, the ratio of the single-layer/few-layer MXene particles having a thickness of 15 nm or less is preferably 90% by volume or more, and more preferably 95% by volume or more.

The metal cation is derived from a metal-containing compound used in the method for producing a two-dimensional particle described later, and the metal cation comprises at least one cation selected from the group consisting of Na and K.

The metal cation preferably is free from Li cations. The phrase “the metal cation is free from Li cations” means that the concentration of Li cation is less than 20 ppm by mass in the total amount of the metal cation as measured by, for example, inductively coupled plasma-atomic emission spectroscopy (ICP-AES).

The metal cation is typically present on the layer. That is, it may be in contact with the layer, or may exist on the layer via another element.

The content of the metal cation in the two-dimensional particle (for example, the total amount of the layer and the metal cation) may be, for example, 20% by mass or less, further 10% by mass or less, particularly 5% by mass or less, and especially 3% by mass or less, and may be, for example, 0.1% by mass or more, and further 0.2% by mass or more.

The content of the metal cation can be measured by, for example, inductively coupled plasma-atomic emission spectroscopy (ICP-AES) or the like.

In the two-dimensional particle, a chlorine atom other than the chlorine atom existing as the modifier or terminal T may exist.

The content of chlorine atoms in the total amount of the layer and the metal cation is 3% by mass or more, preferably 3.3% by mass or more, more preferably 3.5% by mass or more, and preferably 7% by mass or less, more preferably 5% by mass or less, and still more preferably 4.5% by mass or less, in the total amount of the layer and the metal cation. The content of chlorine atoms in the two-dimensional particle is the content of chlorine atoms contained in the layer and the metal cation, and even when the two-dimensional particle and other materials are mixed, chlorine atoms contained in components other than the layer and the metal cation are not included in the content.

The content of chlorine atoms contained in the two-dimensional particle can be measured by combustion ion chromatography.

In the present description, when an “atom” is mentioned for a certain element, the oxidation number of the element is not limited to 0, and may be any number within the range of the oxidation number that the element may have.

In the two-dimensional particle, the content of Li is suppressed. When the two-dimensional particle is used, a conductive film capable of maintaining a high conductivity even under a high humidity condition, for example, a condition with a relative humidity of 99% is provided. The content of Li in the two-dimensional particle (for example, the total amount of the layer and the metal cation) is less than 0.002% by mass, preferably 0.001% by mass or less, and more preferably 0.0001% by mass or less.

The content of Li can be measured by, for example, inductively coupled plasma-atomic emission spectroscopy (ICP-AES) or the like. The detection limit of Li measured by ICP-AES is 0.0001% by mass.

The two-dimensional particle of the present embodiment is free from amines. As described in Non-Patent Literature 2, when delamination treatment of MXene is performed using TMAOH, single-layer MXene is obtained, but TMAOH remains on a surface of the MXene layer even after washing, and the conductivity is lowered due to this. Although TMAOH can be removed in a high temperature state of not lower than 250° C. and not higher than 500° C., MXene may be oxidized and decomposed in the high temperature state. Contrarily, the two-dimensional particle of the present embodiment is not one produced with use of TMAOH for the delamination treatment of MXene and is free from amines. The expression “free from amines” in the present description means that triethylamine (m/z=42, 53, 54) derived from TMAOH is 10 ppm by mass or less as measured using a gas chromatography mass spectrometry (GC-MS) device.

In the present description, the two-dimensional particle refers to a particle having a ratio of (average value of lengths of major axes of two-dimensional planes of two-dimensional particles)/(average value of thicknesses of two-dimensional particles) of 1.2 or more, preferably 1.5 or more, and more preferably 2 or more. The average value of the lengths of the major axes of the two-dimensional planes of the two-dimensional particles and the average value of the thicknesses of the two-dimensional particles may be determined by the methods described later.

### (Average Value of Lengths of Major Axes of Two-Dimensional Planes of Two-Dimensional Particles)

In the two-dimensional particle of the present embodiment, the average value of the lengths of the major axes of the two-dimensional planes is not less than 1 μm and not more than 20 μm. Hereinafter, the average value of the lengths of the major axes of the two-dimensional planes is sometimes referred to as “average flake size”.

The larger the average flake size becomes, the higher the conductivity of the conductive film becomes. Since the two-dimensional particle of the present embodiment has a large average flake size of 1.0 μm or more, a film formed using the two-dimensional particle, for example, a film obtained by laminating the two-dimensional particles achieves a conductivity of 2,000 S/cm or more. The average value of the lengths of the major axes of the two-dimensional planes is preferably 1.5 μm or more, and more preferably 2.5 μm or more. In Non-Patent Literature 3, delamination of MXene is performed by subjecting the MXene to ultrasonic treatment, and a film formed of the single-layer MXene obtained in Non-Patent Literature 3 is expected to be low in conductivity because most of the MXene is reduced in diameter to about several hundred nm in major axis by ultrasonic treatment.

The average value of the lengths of the major axes of the two-dimensional planes is 20 μm or less, preferably 15 μm or less, and more preferably 10 μm or less from the viewpoint of dispersibility in a solution.

As shown in Examples described later, the major axis of a two-dimensional plane refers to a major axis derived by approximating each MXene particle as an elliptical shape in an electron micrograph, and the average value of the lengths of the major axes of the two-dimensional planes refers to a number average of the lengths of the major axes of 80 or more particles. As the electron microscope, a scanning electron microscope (SEM) photograph or a transmission electron microscope (TEM) photograph can be used.

The average value of the lengths of the major axes of the two-dimensional particles of the present embodiment may be measured by dissolving a conductive film comprising the two-dimensional particles in a solvent and dispersing the two-dimensional particles in the solvent. Alternatively, it may be measured from an SEM image of the conductive film.

### (Average Value of Thicknesses of Two-Dimensional Particles)

The average value of the thicknesses of the two-dimensional particles of the present embodiment is preferably not less than 1 nm and not more than 10 nm. The thickness is preferably 7 nm or less, and more preferably 5 nm or less. On the other hand, considering the thickness of single-layer MXene particles, the lower limit of the thickness of the two-dimensional particle may be 1 nm.

The average value of the thicknesses of the two-dimensional particles is determined as a number average dimension (for example, a number average of at least 40 particles) based on an atomic force microscope (AFM) photograph or a transmission electron microscope (TEM) photograph.

### Embodiment 2: Method for Producing Two-Dimensional Particle

Hereinafter, a method for producing a two-dimensional particle according to one embodiment of the present invention will be described in detail, but the present invention is not limited to such an embodiment.

The method for producing a two-dimensional particle according to the present embodiment comprises:
(a) preparing a precursor represented by:

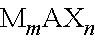

wherein M is at least one metal of Group 3, 4, 5, 6, or 7,
X is a carbon atom, a nitrogen atom, or a combination thereof,
A is at least one element of Group 12, 13, 14, 15, or 16,
n is not less than 1 and not more than 4, and
m is more than n but not more than 5,
(b) removing at least a part of A atoms from the precursor using an etching liquid to produce an etched product, wherein a concentration of chlorine atoms in the etching liquid is 10 mol/L or more;
(c) washing the etched product with water to produce washed product;
(d) mixing the water washed product with a metal-containing compound to produce an intercalated product, wherein the metal-containing compound comprises at least one metal selected from the group consisting of Na and K;
(e) stirring the intercalated product to obtain a delaminated product; and
(f) washing the delaminated product with water to obtain a two-dimensional particle.

Usually, when intercalation treatment is performed using a metal-containing compound containing a cation of metal in the third to fifth periods of the periodic table of the elements, delamination hardly proceeds because the hydration enthalpy of such a metal cation is lower than that of Li ion. However, according to the studies by the present inventors, even when a metal compound containing a metal cation other than a Li ion is used, by setting the concentration of chlorine atoms in an etching liquid to 10 mol/L or more, water easily enters between layers, so that delamination can sufficiently proceed. Although not limited to a specific theory, this is considered to be because the steric effect of chlorine atoms is sufficiently exhibited in the etching liquid.

In the following, the respective steps are described in detail.
Step (a)

First, a prescribed precursor is prepared. The prescribed precursor that can be used in the present embodiment is a MAX phase that is a precursor to MXene, and is represented by a formula below:

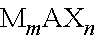

wherein M is at least one metal of Group 3, 4, 5, 6, or 7,
X is a carbon atom, a nitrogen atom, or a combination thereof,
A is at least one element of Group 12, 13, 14, 15, or 16,
n is not less than 1 and not more than 4, and
m is more than n but not more than 5.

Said M, said X, said n, and said m are as described in the first embodiment. A is at least one element of Group 12, 13, 14, 15, or 16, and is usually a Group A element, typically a Group IIIA element or a Group IVA element, and more specifically may comprise at least one element selected from the group consisting of Al, Ga, In, Tl, Si, Ge, Sn, Pb, P, As, S, and Cd, and is preferably Al.

The MAX phase has a crystal structure in which a layer constituted of A atoms is located between two layers each represented by MₘXₙ (each layer can have a crystal lattice in which each X is located in an octahedral array of M). When typically m=n+1, but not limited thereto, the MAX phase includes repeating units in which each one layer of X atoms is located in between adjacent layers of n+1 layers of M atoms (these are also collectively referred to as an “MₘXₙ layer”), and a layer of A atoms (“A atom layer”) is located as a layer next to the (n+1)th layer of M atoms.

The MAX phase can be produced by a known method. For example, a TiC powder, a Ti powder, and an Al powder are mixed in a ball mill, and the resulting mixed powder is calcined under an Ar atmosphere to afford a calcined body (block-shaped MAX phase). Thereafter, the calcined body obtained is pulverized with an end mill to afford a powdery MAX phase for the next step.

A material with a similarly layered structure as MAX may be used as a precursor. Examples of such materials include Zr₂Al₃C₄, Zr₃Al₃C₅, Zr₄ (AlC₂)₃, Zr₂Al₄C₅, Zr₂Al₃C₄, Zr₃Al₃C₅ and Zr₂Al₃C₅.
Step (b)

In the step (b), an etching treatment for removing at least a part of the A atoms from the precursor is performed using an etching liquid.

The etching liquid comprises chlorine atoms. The concentration of chlorine atoms in the etching liquid is 10 mol/L or more, preferably 10.5 mol/L or more, and may be, for example, 20 mol/L or less, or 15 mol/L or less.

The etching liquid preferably comprises HCl, and may further comprise HF. The concentration of HCl in the etching liquid is 10 mol/L or more, preferably 10.5 mol/L or more, and may be, for example, 20 mol/L or less, or 15 mol/L or less. The concentration of HF in the etching liquid is preferably 10% by mass or less, more preferably 6% by mass or less, and is 0% by mass or more, and may be, for example, 1% by mass or more, or 3% by mass or more.

It is preferable that the etching liquid is free from lithium atoms. The expression “free from Li atoms” mentioned for the etching liquid means that the Li concentration in the etching liquid is less than 20 ppm by mass as measured by, for example, combustion ion chromatography.

As the etching operation and other conditions using the etching liquid, conventionally performed conditions can be adopted.
Step (c)

The etched product obtained by the etching treatment is washed with water. By performing water washing, the acid and the like used in the etching treatment can be sufficiently removed. The amount of water to be mixed with the etched product and the washing method are not particularly limited. For example, addition of water, followed by stirring, centrifugation, or the like may be performed. Examples of the stirring method include stirring using a handshaking, an automatic shaker, a share mixer, a pot mill, or the like. The degree of stirring such as the stirring speed and the stirring time may be adjusted according to the amount, concentration, and the like of an acid-treated product to be treated. The washing with water may be performed once or more. Preferably, washing with water is performed a plurality of times. Specifically, for example, steps (i) to (iii), namely, (i) adding water (to the etched product or the remaining precipitate obtained in the following (iii)) and stirring, (ii) centrifuging the stirred product, and (iii) discarding the supernatant after the centrifugation are performed within a range of not less than 2 times and, for example, not more than 15 times.
Step (d)

Intercalation treatment comprising a step of mixing the water washed product obtained by the water washing with a metal-containing compound comprising a metal cation is performed. As a result, the metal cation is intercalated between layers.

The metal cation comprises at least one selected from the group consisting of a Na cation and a K cation.

Examples of the metal-containing compound comprising a metal cation include ionic compounds in which the metal cation is bonded to a cation or an anion. Examples of the metal-containing compound include an iodide, a phosphate, a sulfide salt including a sulfate, a nitrate, an acetate, and a carboxylate of the metal cation.

The content of the metal-containing compound in the formulation for intercalation treatment including the metal-containing compound is preferably 0.001% by mass or more, more preferably 0.01% by mass or more, and still more preferably 0.1% by mass or more. From the viewpoint of dispersibility in a solution, the content of the metal-containing compound in the formulation for intercalation treatment is preferably 10% by mass or less, and more preferably 18 by mass or less.

It is preferable the formulation for that intercalation treatment is free from lithium atoms. The expression “free from Li atoms” mentioned for the formulation for intercalation treatment means that the Li concentration in the formulation for intercalation treatment is, for example, than 20 ppm by mass as measured by combustion ion chromatography.

A specific method of the intercalation treatment is not particularly limited, and for example, the metal-containing compound may be mixed with the water washed product, followed by stirring or leaving to stand. For example, stirring at room temperature may be performed. Examples of the stirring method include a method using a stirring bar such as a stirrer, a method using a stirring blade, a method using a mixer, a method using a centrifugal device, and the like, and the stirring time may be set according to the production scale of the single-layer/few-layer MXene particles, and the stirring time may be set, for example, between 12 and 24 hours.
Step (e)

In the step (e), delamination treatment comprising a step of stirring the intercalated product obtained by performing the intercalation treatment is performed. By this delamination treatment, the MXene particles can be made into a single layer or fewer layers.

Conditions for the delamination treatment are not particularly limited, and the delamination treatment may be performed by a known method. Examples of the stirring method include stirring using an ultrasonic treatment, handshaking, an automatic shaker or the like. The degree of stirring such as the stirring speed and the stirring time may be adjusted according to the amount, concentration, and the like of a treated product to be treated. For example, the slurry resulting from the intercalation is centrifuged to discard the supernatant, and then pure water is added to the remaining precipitate, and then stirring is performed, for example, by handshaking or using an automatic shaker, followed by layer separation (delamination). The removal of the undelaminated substance may comprise a step of performing centrifugal separation to discard the supernatant, and then washing the remaining precipitate with water. For example, (i) pure water is added to the remaining precipitate resulting from the discarding of a supernatant and stirred, (ii) centrifugation is performed, and (iii) a supernatant is recovered. This operation of (i) to (iii) may be repeated once or more, preferably not less than twice and not more than ten times to obtain a supernatant containing single-layer/few-layer MXene particles as a delaminated product. Alternatively, by centrifuging the supernatant, followed by discarding the supernatant resulting from the centrifugation, a clay containing single-layer/few-layer MXene particles as a delaminated product may be obtained.

In the production method of the present embodiment, ultrasonic treatment may not be performed as delamination. When the ultrasonic treatment is not performed, particle breakage hardly occurs, and single-layer/few-layer MXene particles having a large plane parallel to the layer of particles, that is, a two-dimensional plane can be obtained easily.

The delaminated product obtained by stirring can be used as received two-dimensional particles containing single-layer/few-layer MXene particles, or may be washed with water, as necessary.

### Embodiment 3: Conductive Film

Examples of the application of the two-dimensional particle of the present embodiment include a conductive film comprising the two-dimensional particle. A conductive film of the present embodiment will be described with reference to FIG. 2. In FIG. 2, a conductive film 30 obtained by stacking only the two-dimensional particles 10 is illustrated, but the conductive film of the present embodiment is not limited thereto. The conductive film may comprise an additive such as a binder added at the time of film formation, as necessary. The proportion of the additive in the conductive film (when dried) is preferably 30% by volume or less, more preferably 10% by volume or less, still more preferably 5% by volume or less, and most preferably 0% by volume.

As a method for producing a conductive film without using the binder or the like, the conductive film can be produced by subjecting the supernatant containing two-dimensional particles obtained by the delamination to suction filtration, or by performing, once or two or more times, a step of spraying a slurry of two-dimensional particles mixed with a dispersion medium in an appropriate concentration and then removing the dispersion medium by drying or the like. The spraying method may be, for example, an airless spraying method or an air spraying method, and specific examples thereof include a method of spraying using a nozzle such as a one-fluid nozzle, a two-fluid nozzle, or an air brush. Examples of the dispersion medium that may be contained in the slurry include water and organic media as N-methylpyrrolidone, N-methylformamide, N, N-dimethylformamide, methanol, ethanol, dimethylsulfoxide, ethylene glycol, and acetic acid.

Examples of the binder include acrylic resin, polyester resin, polyamide resin, polyolefin resin, polycarbonate resin, polyurethane resin, polystyrene resin, polyether resin, and polylactic acid.

The conductivity of the conductive film is preferably 2,000 S/cm or more and more preferably 5,000 S/m or more, and may be, for example, 100,000 S/cm or less, or 50,000 S/cm or less.

The conductivity of the conductive film of the present embodiment is determined by substituting the thickness of the conductive film and the surface resistivity of the conductive film measured by a four-probe method into the following formula.

Conductivity [S/cm]=1/(thickness [cm] of conductive film×surface resistivity [Q/square] of conductive film)

Other applications using the two-dimensional particle of the present embodiment may be a conductive paste comprising the two-dimensional particle, and a conductive composite comprising the two-dimensional particle and a resin. They are also suitable for applications requiring high conductivity and few decrease in conductivity even under high humidity conditions.

Examples of the resin that can be contained in the conductive paste and the conductive composite include the same resins as the resins that can be contained in the conductive film. Examples of the dispersion medium that can be contained in the conductive paste include water and organic media such as N-methylpyrrolidone, N-methylformamide, N, N-dimethylformamide, methanol, ethanol, dimethylsulfoxide, ethylene glycol, and acetic acid.

The conductive film of the present embodiment can be used for any suitable application. For example, the conductive film can be used for an application in which it is required to be able to maintain a high conductivity even under high humidity conditions, such as an electrode or an electromagnetic shield (EMI shield) in any appropriate electric device.

The electrode is not particularly limited, and may be, for example, a capacitor electrode, a battery electrode, a bioelectrode, a sensor electrode, or an antenna electrode. By using the conductive film of the present embodiment, a capacitor and a battery both having a large capacity, a bioelectrode having a low impedance, and a sensor and an antenna both having a high sensitivity even with a smaller volume (device-occupied volume) are obtained.

The capacitor may be an electrochemical capacitor. The electrochemical capacitor is a capacitor utilizing a capacitance developed due to a physicochemical reaction between an electrode (electrode active material) and ions (electrolyte ions) in an electrolytic solution, and can be used as a device (power storage device) that stores electric energy. The battery may be a repeatedly chargeable and dischargeable chemical battery. The battery may be, for example, but is not limited to, a lithium ion battery, a magnesium ion battery, a lithium sulfur battery, or a sodium ion battery.

The bioelectrode is an electrode for acquiring a biological signal (i.e., a biological signal sensing electrode). The bioelectrode may be, for example, but is not limited to, an electrode for measuring EEG (electroencephalogram), ECG (electrocardiogram), EMG (electromyogram), or EIT (electrical impedance tomography).

The sensor electrode is an electrode for detecting a target substance, state, abnormality, or the like. The sensor may be, for example, but is not limited to, a gas sensor, a biosensor (i.e., a chemical sensor using a molecular recognition mechanism of biological origin).

The antenna electrode is an electrode for emitting an electromagnetic wave into and/or space receiving an electromagnetic wave in space.

### Embodiment 4: Adsorbent

Examples of the application of the two-dimensional particle of the present embodiment include an adsorbent comprising a two-dimensional particle. Since the two-dimensional particle has a controlled content of Li, the two-dimensional particle can also be used for applications in which biocompatibility is required, such as a separation membrane in an artificial dialysis device.

The adsorbent may be constituted of only two-dimensional particles, and may further comprise one or more materials selected from among ceramics, metal, and resin, as necessary. By employing a composite made of the two-dimensional particle of the present embodiment and one or more materials selected from among ceramics, metal, and resin, an adsorbent that stably exhibits adsorption performance, for example, performance of adsorbing urea is provided. The ceramics, the metal, and the resin can be introduced during the production of the adsorbent.

Examples of the ceramics include metal oxides such as silica, alumina, zirconia, titania, magnesia, cerium oxide, zinc oxide, barium titanate-based ceramics, hexaferrite, and mullite; and non-oxide ceramics such as silicon nitride, titanium nitride, aluminum nitride, silicon carbide, titanium carbide, tungsten carbide, boron carbide, and titanium boride. Examples of the metal include iron, titanium, magnesium, aluminum, and alloys based on these metals.

Examples of the resin include cellulose resins and synthetic resins. The resin is preferably a hydrophilic resin. The hydrophilic resin can be prepared by blending a hydrophilic aid into a hydrophobic resin (a resin having no hydrophilicity) or subjecting a hydrophobic polymer to hydrophilization treatment. The resin (preferably a hydrophilic resin) more preferably contains one or more materials selected from the group consisting of polysulfone, cellulose acetate, regenerated cellulose, polyethersulfone, water-soluble polyurethane, polyvinyl alcohol, sodium alginate, a water-soluble acrylic acid-based polymer, polyacrylamide, polyaniline sulfonic acid, and nylon.

The hydrophilic resin is preferably, for example, a hydrophilic polymer having a polar group, in which the polar group is a group that may form a hydrogen bond with a modifier or terminal T of the layer. Examples of the hydrophilic resin include water-soluble polyurethane, polyvinyl alcohol, sodium alginate, a water-soluble acrylic acid-based polymer, polyacrylamide, polyaniline sulfonic acid, and nylon, and water-soluble polyurethane, polyvinyl alcohol, and sodium alginate are more preferable, and water-soluble polyurethane is still more preferable.

The hydrophilic resin preferably exhibits biocompatibility. Examples of the biocompatible resin include resins for hemodialysis and hemofiltration. Specific examples thereof include polymethyl methacrylate, polyacrylonitrile, cellulose, cellulose acetate, polysulfone, polyvinyl alcohol, and a vinyl alcohol copolymer such as a copolymer of polyvinyl alcohol and ethylene. Examples of the biocompatible resin preferably include polysulfone, polymethyl methacrylate, and cellulose acetate, and more preferably include polysulfone and polymethyl methacrylate.

The content of the resin contained in the composite may be appropriately set according to the application, and may be more than 0% by volume and, for example, not more than 80% by volume, or not more than 50% by volume, or especially not more than 30% by volume, or particularly not more than 10% by volume, or not more than 5% by volume, in terms of the proportion in the adsorbent (when dried).

The method for producing an adsorbent formed of the composite is not particularly limited. As one embodiment, a sheet-shaped adsorbent containing a resin can be produced by mixing two-dimensional particles and a resin to form a slurry, applying the slurry to a substrate (for example, a board) to form a coating film, and, as necessary, drying and/or curing the coating film.

The two-dimensional particles may be mixed as received with the resin, or may be dispersed in a dispersion medium to form a dispersion and then mixed with the resin. The dispersion medium is typically water, and may further comprise a relatively small amount of other liquid substance in some cases. The content of the other liquid substance may be, for example, 30% by mass or less, and may be 20% by mass or less in the dispersion medium.

The two-dimensional particles and the resin can be mixed using a dispersing device such as a homogenizer, a propeller stirrer, a thin-film spin type stirrer, a planetary mixer, a mechanical shaker, or a vortex mixer.

The method for applying the slurry to the substrate is not limited, and examples thereof include a method in which spray application is performed using an airless spray, an air spray, or the like, specifically, spray application using a nozzle such as a one-fluid nozzle, a two-fluid nozzle, or an air brush; slit coating using a table coater, a comma coater, or a bar coater; a printing method such as screen printing and metal mask printing; spin coating; immersion; dripping; a brush; a roller; a roll coater; a curtain flow coater; a roller curtain coater; a die coater; and an application method by electrostatic coating.

Drying and/or curing may be performed, for example, at a temperature of 400° C. or lower using a normal pressure oven or a vacuum oven.

The application and drying may be repeated multiple times as necessary until a sheet (film) having a desired thickness is obtained.

As another embodiment, the adsorbent comprising ceramic or metal can be produced by, for example, mixing two-dimensional particles with, for example, particulate ceramics or metal, and heating the mixture at a low temperature at which the composition of the two-dimensional particles can be maintained.

The shape of the adsorbent of the present embodiment is not limited. The shape of the adsorbent may be a shape having a thickness, a rectangular parallelepiped, a sphere, a polygonal body, or the like besides the case of having a sheet-like form such as the above-described film.

### (Adsorbent Sheet)

Preferred embodiments of the adsorbent of the present embodiment include an adsorbent sheet. The adsorbent sheet may be the adsorbent of the present embodiment, that is, an adsorbent sheet formed of two-dimensional particles or a composite comprising the two-dimensional particles, or alternatively may be a product in which the adsorbent of the present embodiment is formed on a substrate surface. The substrate may be formed of one or more materials selected from among the above-described ceramics, metal, and resin. Among them, the substrate is preferably an adsorbent sheet in which the adsorbent of the present embodiment is formed on a substrate formed of the resin described above. The adsorbent may be formed on a part of the surface of the substrate or may be formed on the entire surface of the substrate. Examples of the method for forming the adsorbent on the substrate include the method described as the method for applying a slurry to a substrate.

### (Application of Adsorbent)

The adsorbent of the present embodiment can be used, for example, for adsorption of a polar organic compound. The polar organic compound is a generic term for organic compounds having polarity, specifically, organic compounds having a polar group. Examples of the polar group include a hydroxyl group (OH group), an NO₂ group, an amino group (NH group, NH₂ group), and a COOH group, and these polar groups can form a hydrogen bond with a hydrogen atom contained in a water molecule. Among the polar organic compounds, examples of the object to be adsorbed include polar solvents such as alcohols having a hydroxyl group, compounds having an amino group, and ammonia, and particularly include compounds having one or more of a hydroxyl group and an amino group, and ammonia.

Among the compounds having one or more of a hydroxyl group and an amino groups, examples of the compound having a hydroxyl group include monohydric alcohols having 1 to 22 carbon atoms; polyhydric phenols; polyhydric alcohols such as ethylene glycol, propylene glycol, and glycerin; alkanolamines such as triethanolamine; and sugars such as xylose and glucose.

Examples of the compound having an amino group include monoamines such as methylamine and dimethylamine; diamine such as ethylenediamine; polyamines such as diethylenetriamine; aromatic amines such as aniline; amino acids such as valine and leucine, urea, uric acid, urates, and creatinine. Examples of the compound having a hydroxyl group and an amino group include ethanolamine and diethanolamine.

The adsorbent of the present embodiment is preferably used for adsorbing uremic toxins including, for example, urea, uric acid, and creatinine. The adsorbent of the present embodiment can be optimally used for adsorbing urea.

The adsorbent of the present embodiment can be used for adsorbing and removing waste products such as urea in hemodialysis, hemofiltration, hemodiafiltration, peritoneal dialysis, and the like. In addition, the adsorbent of the present embodiment can be used in an artificial dialysis machine for performing the hemodialysis, hemofiltration, hemodiafiltration, peritoneal dialysis, and the like. The form of the adsorbent is not particularly limited, and may be, for example, a porous type, a hollow fiber type, or a flat membrane laminate type.

Although the two-dimensional particle in one embodiment of the present invention has been described in detail above, various modifications may be made. The two-dimensional particle of the present invention may be produced by methods different from the production methods in the above embodiments. It should be noted that the methods for producing the two-dimensional particle of the present invention are not limited only to those that provide the two-dimensional particle according to the above embodiments.

### EXAMPLES

### Examples 1 to 4, Comparative Example 1

In Examples 1 to 4 and Comparative Example 1, (1) preparation of a precursor (MAX), (2) etching of the precursor, (3) washing, (4) intercalation of a metal cation, (5) delamination, and (6) water washing each described in detail below were performed in order, affording two-dimensional particles.

### (1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was calcined in an Ar atmosphere at 1350° ° C. for 2 hours. The calcined body (block) obtained was pulverized with an end mill to a maximum size of 40 μm or less. Thereby, Ti₃AlC₂ powder was obtained as a precursor (MAX).

### (2) Etching of Precursor

Using the Ti₃AlC₂ particles (powder) prepared by the above method, etching was performed under the following etching conditions, affording a solid-liquid mixture (slurry) containing a solid component derived from the Ti₃AlC₂ powder.

### (Etching Conditions)

Precursor: Ti₃AlC₂ (sieved with a mesh size of 45 μm)
See Table 1 for etching liquid composition
Amount of precursor input: 3.0 g
Etching container: 100 mL IBOY
Etching temperature: 35° C.
Etching time: 24 hours
Stirrer rotation speed: 400 rpm

### (3) Washing

The slurry was divided into two portions, each of which was inserted into two 50 mL centrifuge tubes, centrifuged under the condition of 3,500 G using a centrifuge, and then the supernatant was discarded. An operation of adding 40 mL of pure water to each centrifuge tube, centrifuging again at 3,500 G for 5 minutes to separate and remove the supernatant was repeated 11 times. After final centrifugation, the supernatant was discarded, affording a Ti₃C₂Tₛ-water medium clay.

### (4) Intercalation of Metal Cations

The metal-containing compound shown in Table 1 was added to the Ti₃C₂Tₛ-water medium clay prepared by the above method, and the mixture was stirred at not lower than 20° C. and not higher than 25° C. for 10 hours, thereby performing intercalation using a metal cation as an intercalator. The detailed conditions of the intercalation are as follows.

### (Conditions of Intercalation)

Ti₃C₂Tₛ-water medium clay (MXene after washing): solid content 0.75 g
See Table 1 for the metal-containing compound and the added amount.
Intercalation container: 100 mL IBOY
Temperature: not lower than 20° C. and not higher than

25° C. (room temperature)
Time: 10 hours
Stirrer rotation speed: 800 rpm

### (7) Delamination

The slurry resulting from the intercalation was charged into a 50 mL centrifuge tube, 20 mL of pure water was added thereto, centrifugation was then performed under the condition of 3,500 G using a centrifuge, and the supernatant was discarded. Subsequently, an operation of adding 40 mL of pure water, stirring the mixture with a shaker for 15 minutes, centrifuging the mixture at 3,500 G, and collecting the supernatant as a single-layer MXene particle-containing liquid was repeated 4 times, affording a single-layer MXene particle-containing supernatant. Furthermore, this supernatant was centrifuged using a centrifuge under the conditions of 4,300 G and 2 hours, and then the supernatant was discarded, affording two-dimensional particles (single-layer MXene particle clay).

TABLE 1

| | | | | | | | Comparative |
|---|---|---|---|---|---|---|---|
| | | | Example 1 | Example 2 | Example 3 | Example 4 | Example 1 |
| Composition | 49% Aqueous | mL | 6 | 6 | 6 | 6 | 6 |
| of etching | HF solution | | | | | | |
| liquid | 12M Aqueous | mL | 54 | 54 | 48 | 48 | 36 |
| | HCl solution | | | | | | |
| | H₂O | mL | | | 6 | 6 | 18 |
| | Chlorine atom | mol/M | 12 | 12 | 11 | 11 | 9 |
| | concentration | | | | | | |
| Metal- | NaCl | g | 1.03 | | 1.03 | | |
| containing | KCl | g | | 1.32 | | 1.32 | |
| compound | | | | | | | |

| | | |
|---|---|---|
| LiCl | g | 0.75 |

### (Method for Producing Conductive Film)

The clays obtained in Examples 1 to 4 and Comparative Example 1 were subjected to suction filtration. After the filtration, vacuum drying was performed at 80° C. for 24 hours, preparing a conductive film comprising two-dimensional particles. As a filter for the suction filtration, a membrane filter (Durapore, manufactured by Merck Corporation, pore size: 0.45 μm) was used. The supernatant contained 0.05 g, in solid content, of two-dimensional particles and 40 mL of pure water.

### (Measurement of Density of Conductive Film)

A conductive film was punched into a disk shape having a diameter of 12 mm with a punch. Then, the mass of the disk was measured with an electronic balance, and the thickness thereof was measured with a height gauge. Then, the density of the conductive film was calculated from these measured values. The result is shown in Table 1.

### (Method for Measuring Conductivity of Conductive Film)

The conductivity of the obtained conductive film containing two-dimensional particles was determined. For the conductivity, the resistivity (Q/square) and the thickness (μm) were measured at three points per sample, the conductivity (S/cm) was calculated from these measured values, and the average value of three conductivities obtained by this calculation was adopted. For resistivity measurement, the surface resistance of the conductive film was measured by a four-terminal method using a simple low resistivity meter (Loresta-AX MCP-T370, manufactured by Mitsubishi Chemical Analytech Co., Ltd.). A micrometer (MDH-25 MB, manufactured by Mitutoyo Corporation) was used for the thickness measurement. Then, a volume resistivity was determined from the obtained surface resistance and the thickness of the conductive film, and the reciprocal of the value was taken to determine a conductivity, which was designated as Eo.

### (Test of Resistance to Moisture Absorption)

The obtained conductive film was placed in a thermo-hygrostat at a relative humidity of 99% and a temperature of 25° C., taken out at predetermined time intervals, and a conductivity was measured and designated as E. A conductivity change rate was calculated by dividing E by Eo.

### (Measurement of Chlorine Concentration in Two-Dimensional Particle)

The chlorine concentration in the two-dimensional particles obtained in Examples 1 to 4 and Comparative Example 1 was measured using a combustion ion chromatography apparatus (Dionex ICS-5000) manufactured by Thermo Fisher Scientific K.K..

### (Method for Detecting Element on Layer Surface)

The obtained conductive film containing two-dimensional particles was measured by X-ray photoelectron spectroscopy (XPS), and an organic low molecular weight compound contained in the two-dimensional particles and an element on a layer surface were detected. For the XPS measurement, Quantum 2000 manufactured by ULVAC-PHI, Inc. was used.

### (Method for Detecting Metal Cation)

The solution obtained by dissolving the obtained two-dimensional particles by an alkali fusion method was measured by inductively coupled plasma-atomic emission spectroscopy (ICP-AES), and metal cations contained in the two-dimensional particles were detected. For the ICP-AES measurement, iCAP 7400 manufactured by Thermo Fisher Scientific K.K. was used.

### (Measurement of Average Value of Lengths of Major Axes of Two-Dimensional Planes of Two-Dimensional Particles)

A slurry in which two-dimensional particles were dispersed in water was applied to an alumina porous substrate and dried, and a scanning electron microscope (SEM) photograph was taken to perform measurement. The magnification was set to 2,000 times, and 80 or more two-dimensional particles (MXene particles) that could be visually confirmed in one or a plurality of SEM image fields (about 1 field to 3 fields) having a field size of 45 μm×45 μm were targeted. The shape of the two-dimensional plane of each two-dimensional particle (MXene particle) (the shape viewed from a direction orthogonal to the layer of each two-dimensional particle) was approximated to an elliptical shape, and the length of the major axis thereof was measured. The average value of the lengths of the major axes measured for the two-dimensional particles (MXene particles) as a target was taken as the average value of the lengths of the major axes of the two-dimensional planes of the two-dimensional particles. SEM image analysis software “A-Zou Kun” (registered trademark, produced by Asahi Kasei Engineering Corporation) was used to approximate the elliptical shape. When a porous substrate is used as the substrate, fine black spots in a micrograph may be derived from the substrate. Therefore, before the image analysis, processing of erasing a porous portion of the background by image processing was performed, as necessary.

The measurement results are shown in Table 2.

TABLE 2

| | | Moisture resistance test | | | | | | | | |
|---|---|---|---|---|---|---|---|---|---|---|
| | | (Conductivity change rate) | | | | | | | Average | |
| | Chlorine atom | 0 | 30 | 60 | 180 | 1440 | Metal cation | | particle | Li |
| | concentration | min | min | min | min | min | Na | K | size | content |
| | (wt %) | (%) | (%) | (%) | (%) | (%) | (wt %) | (wt %) | (μm) | (wt %) |
| Example 1 | 3.9 | 100 | 100 | 69 | 69 | 64 | 0.68 | | 3.3 | <0.002 |
| Example 2 | 4.2 | 100 | 93 | 73 | 75 | 66 | | 1.77 | 3.5 | <0.002 |
| Example 3 | 3.7 | 100 | 84 | 81 | 81 | 74 | 0.46 | | 3.9 | <0.002 |
| Example 4 | 3.6 | 100 | 85 | 87 | 87 | 57 | | 1.62 | 3.1 | <0.002 |
| Comparative | 2.7 | 100 | 81 | 51 | 55 | 47 | | | 3.2 | 2.7 |
| Example 1 | | | | | | | | | | |

From the results in Table 2 above, the MXene two-dimensional particles obtained in the present embodiment was free from Li, and the decrease in conductivity was suppressed even when the MXene two-dimensional particles were placed under high humidity conditions for a long time. In addition, in the MXene two-dimensional particles obtained in the present embodiment, the average value of the lengths of the major axes of the two-dimensional planes was 1 μm or more, and the average value of the thicknesses was 10 nm or less. Further, a film (conductive film) was obtained without adding a binder by using the MXene two-dimensional particles obtained in the present embodiment.

Contrary to this, in Comparative Example 1, since Li was used as an intercalator, the electrical conductivity was significantly when placed under a high humidity condition.

The two-dimensional particle, the conductive film, and the conductive paste of the present invention can be used in any suitable application, and can be particularly, preferably used, for example, as electrodes in electrical devices.

### REFERENCE SIGNS LIST

1a, 1b Layer body (MₘXₙ layer)
3a, 5a, 3b, 5b Modifier or terminal T
7a, 7b MXene layer
10, 10a, 10b MXene particles (particles of layered material)
30 Conductive film

1. A two-dimensional particle comprising:
one or plural layers, the one or plural layers comprising a layer body represented by: 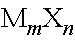
wherein M is at least one metal of Group 3, 4, 5, 6, or 7,
X is a carbon atom, a nitrogen atom, or a combination thereof,
n is not less than 1 and not more than 4,
m is more than n but not more than 5, and
a modifier or terminal T existing on a surface of the layer body, wherein T is at least one selected from the group consisting of a hydroxyl group, an amine group, a fluorine atom, a chlorine atom, a bromine atom, an iodine atom, an oxygen atom, a sulfur atom, a selenium atom, a tellurium atom and a hydrogen atom; and
a metal cation comprising at least one cation selected from the group consisting of Na and K, wherein
a content of Li in the two-dimensional particle is less than 0.002% by mass.

2. The two-dimensional particle according to claim 1, wherein a content of chlorine atoms in the two-dimensional particle measured by combustion ion chromatography is 3% by mass or more in a sum of the layer and the metal cation.

3. The two-dimensional particle according to claim 1, wherein a total content of Na and K is not less than 0.1% by mass and not more than 10% by mass.

4. The two-dimensional particle according to claim 1, wherein an average value of lengths of major axes of two-dimensional planes of the two-dimensional particle is not less than 1 μm and not more than 20 μm.

5. The two-dimensional particle according to claim 1, wherein an average thickness of the two-dimensional particle is not less than 1 nm and not more than 10 nm.

6. The two-dimensional particle according to claim 1,
wherein the metal cation is on the one or more layers.

7. The two-dimensional particle according to claim 1,
wherein a content of the metal cation in the two-dimensional particle is 20 mass % or less.

8. A conductive film comprising the two-dimensional particle according to claim 1.

9. The conductive film according to claim 8, wherein a conductivity of the conductive film is 2,000 S/cm or more.

10. A conductive paste comprising the two-dimensional particle according to claim 1 and a dispersion medium.

11. A conductive composite comprising the two-dimensional particle according to claim 1 and a resin.

12. An electromagnetic shield comprising the two-dimensional particle according to claim 1.

13. An adsorbent comprising the two-dimensional particle according to claim 1.

14. A bioelectrode comprising the two-dimensional particle according to claim 1.

15. A method for producing a two-dimensional particle, the method comprising:
(a) preparing a precursor represented by: 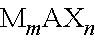
wherein M is at least one metal of Group 3, 4, 5, 6, or 7,
X is a carbon atom, a nitrogen atom, or a combination thereof,
A is at least one element of Group 12, 13, 14, 15, or 16,
n is not less than 1 and not more than 4, and
m is more than n but not more than 5;
(b) removing at least a part of A atoms from the precursor using an etching liquid to produce an etched product, wherein a concentration of chlorine atoms in the etching liquid is 10 mol/L or more;
(c) washing the etched product with water to produce washed product;
(d) mixing the water washed product with a metal-containing compound to produce an intercalated product, wherein the metal-containing compound comprises at least one metal selected from the group consisting of Na and K;
(e) stirring the intercalated product to obtain a delaminated product; and
(f) washing the delaminated product with water to obtain a two-dimensional particle.

16. The method for producing a two-dimensional particle according to claim 15, wherein a content of the metal-containing compound in a formulation for producing the intercalated product is 0.001% by mass or more.
